(19) United States
(12) Patent Application Publication
(10) Pub. No.: US 2006/0034704 A1
(43) Pub. Date: Feb. 16, 2006

(54) Multi-cylinder, reciprocating compressors for air conditioning systems mounted in vehicles

(76) Inventors: Hiroshi Baba, Isesaki-shi (JP); Yoshinobu Ichikawa, Gunma-gun (JP)

Correspondence Address: BAKER BOTTS LLP;C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 11/199,139

(22) Filed: Aug. 9, 2005

(30) Foreign Application Priority Data
Aug. 16, 2004 (JP) 2004-236332

(51) Int. Cl. F04B 27/08; A61M 1/00

(52) U.S. Cl. 417/269; 417/437

(57) ABSTRACT

A multi-cylinder reciprocating compressor for an air conditioning system mounted in a vehicle includes a columnar cylinder block with cylinder bores, a front head provided at one side of the cylinder block to form a crank chamber, a rear head provided at the other side of the cylinder block, a valve plate interposed between the cylinder block and the rear head, a discharge chamber formed in the rear head, and a suction chamber formed in the rear head having an annular shape and positioned radially outside of the discharge chamber. The suction chamber is divided into first and second, annular chambers by a partition wall extending in a circumferential direction. As a result of this configuration, the pulsation of refrigerant generated in the annular suction chamber may be reduced, and the vibration and noise of the air conditioning system may be reduced.

### BACKGROUND OF THE INVENTION

1. Field of the Invention

The present invention relates to a multi-cylinder, reciprocating compressor for an air conditioning system mounted in a vehicle.

2. Description of Related Art

A multi-cylinder, reciprocating compressor for an air conditioning system mounted in a vehicle may comprise a columnar cylinder block with a plurality of cylinder bores formed therethrough at intervals in a circumferential direction; a front head formed in a cup-like shape, which confronts one end surface of the cylinder block to form a crank chamber; a rear head formed in a cup-like shape, which confronts the other end surface of the cylinder block to form therein a suction chamber and a discharge chamber; a drive shaft extending in the crank chamber and through a bottom wall of the front head; and a valve plate interposed between the cylinder block and the rear head. Such a compressor is disclosed, for example, in Japanese Patent Application No. JP-A-11-173262. The suction chamber communicates with a suction port formed through an outer wall of the rear head and with each of the cylinder bores through a suction valve and a suction hole formed through the valve plate, and the discharge chamber communicates with a discharge port formed through the outer wall of the rear head and with each of the cylinder bores through a discharge valve and a discharge hole formed through the valve plate. In such a known compressor, the suction chamber is formed in an annular shape at a position radially outside of the discharge chamber.

In such a structure of the suction chamber, however, refrigerant gas drawn into the annular suction chamber may generate a pulsation at a frequency, the wavelength of which is a circumferential length of the annular shape of the suction chamber. The pulsation may be transferred outside of the compressor through the suction port, and the transferred pulsation may cause vibration and noise in an air conditioning system connected to the compressor through a refrigerant circuit.

### SUMMARY OF THE INVENTION

Accordingly, it is an object of the present invention to provide a multi-cylinder, reciprocating compressor for an air conditioning system mounted in a vehicle, which suppresses the above-described vibration and noise of the air conditioning system due to a pulsation of refrigerant gas in an annular suction chamber.

To achieve the foregoing and other objects, a multi-cylinder, reciprocating compressor for an air conditioning system mounted in a vehicle, according to the present invention, comprises a columnar cylinder block comprising a plurality of cylinder bores formed therein at intervals in a circumferential direction; a first (e.g., front) head formed as a cup-like shape, which confronts one end surface of the cylinder block to form a crank chamber; a second (e.g., rear) head formed as a cup-like shape, which confronts the other end surface of the cylinder block; a drive shaft extending in the crank chamber and through a first (e.g., bottom) wall of the first head; a valve plate interposed between the cylinder block and the second head; a discharge chamber formed in the second head and in fluid communication with a discharge port formed on a second (e.g., outer) wall of the second head and with each of the cylinder bores through a discharge valve and a discharge hole formed on the valve plate; and a suction chamber disposed in the second head having an annular shape and disposed radially outside of the discharge chamber, which is in fluid communication with a suction port formed through the second wall of the second head and with each of the cylinder bores through a suction valve and a suction hole formed through the valve plate. The suction chamber comprises a first chamber and a second chamber. The second chamber is disposed radially inside of the first chamber. The first and the second chambers are separated by a suction chamber partition wall extending in a circumferential direction.

In the multi-cylinder, reciprocating compressor, according to the present invention, the annular suction chamber is divided into two chambers: a radially exterior, first chamber and a radially interior, second chamber, and the circumferential lengths of the first and second chambers may be different from each other. Consequently, the frequency of the pulsation of refrigerant generated in the suction chamber is dispersed between two frequency values separated from each other as compared with a single frequency value in the known structure of a single, annular suction chamber. By changing the pulsation in the suction chamber from a pulsation with a single frequency value to a pulsation with two frequency values, the vibration level of the pulsation may be reduced, the levels of the vibration and noise of an air conditioning system ascribed to the pulsation may be reduced, and, ultimately, the noise of the air conditioning system as a whole may be reduced.

In a preferred embodiment of the multi-cylinder, reciprocating compressor, according to the present invention, the suction chamber partition wall may comprise a plurality of partial partition walls extending in the direction of the suction chamber partition wall. By dividing the suction chamber partition wall into a plurality of partial partition walls in the direction in which the wall extends, a plurality of propagation routes for the pulsation directed to the suction port are formed. Thus, a condition similar to the condition in which many suction chambers with different circumferential lengths are formed, may be created Consequently, the frequency of the pulsation of refrigerant generated in the suction chamber is separated into a plurality of frequency values as compared with a single frequency value in the known structures having a single, annular suction chamber. By changing the pulsation in the suction chamber from a pulsation with a single frequency value to a pulsation with a plurality of frequency values, the vibration level of the pulsation may be reduced further, the levels of the vibration and noise of an air conditioning system ascribed to the pulsation may be reduced further, and, ultimately, the noise of the air conditioning system as a whole may be reduced further.

Further, in a preferred embodiment of the multi-cylinder, reciprocating compressor, according to the present invention, the plurality of partial partition walls is an uneven number of partial partition walls. When the suction chamber partition wall is divided into a plurality of partial partition walls in the direction in which the wall extends, a division into an uneven number may achieve a more effective reduction of the vibration level of the pulsation than a division into an even number.

The division of the suction chamber partition wall into a plurality of partial partition walls may be accomplished, for example, by disposing each end of each of the plurality of partial partition walls at an edge of the suction hole.

In the above-described multi-cylinder, reciprocating compressor, according to the present invention, the annular suction chamber is divided into two chambers: the radially exterior, first chamber and the radially interior, second chamber, and the circumferential lengths of the first and the second chambers are different from each other. Consequently, the frequency of the pulsation of refrigerant generated in the suction chamber is dispersed between two frequency values separated from each other (preferably, more than three frequency values) as compared with a single frequency value in the known structure of a single, annular suction chamber. By changing the pulsation in the suction chamber from a pulsation with a single frequency value to a pulsation with two frequency values (preferably, more than three frequency values), the level of the pulsation may be reduced significantly, the levels of the vibration and noise of an air conditioning system ascribed to the pulsation may be reduced significantly, and, ultimately, the noise of the air conditioning system as a whole may be reduced significantly.

Further objects, features, and advantages of the present invention will be understood from the following detailed description of preferred embodiments of the present invention with reference to the accompanying figures.

### BRIEF DESCRIPTION OF THE DRAWINGS

Embodiments of the invention now are described with reference to the accompanying figures, which are given by way of example only, and are not intended to limit the present invention.

FIG. 1 is a cross-sectional view of a multi-cylinder, reciprocating compressor according to an embodiment of the present invention.

FIG. 2 is a cross-sectional view of the multi-cylinder reciprocating compressor depicted in FIG. 1, as viewed along line II-II of FIG. 1.

FIG. 3 is a cross-sectional view of a multi-cylinder, reciprocating compressor according to another embodiment of the present invention, showing a view corresponding to the view depicted in FIG. 2.

### DETAILED DESCRIPTION OF PREFERRED EMBODIMENTS

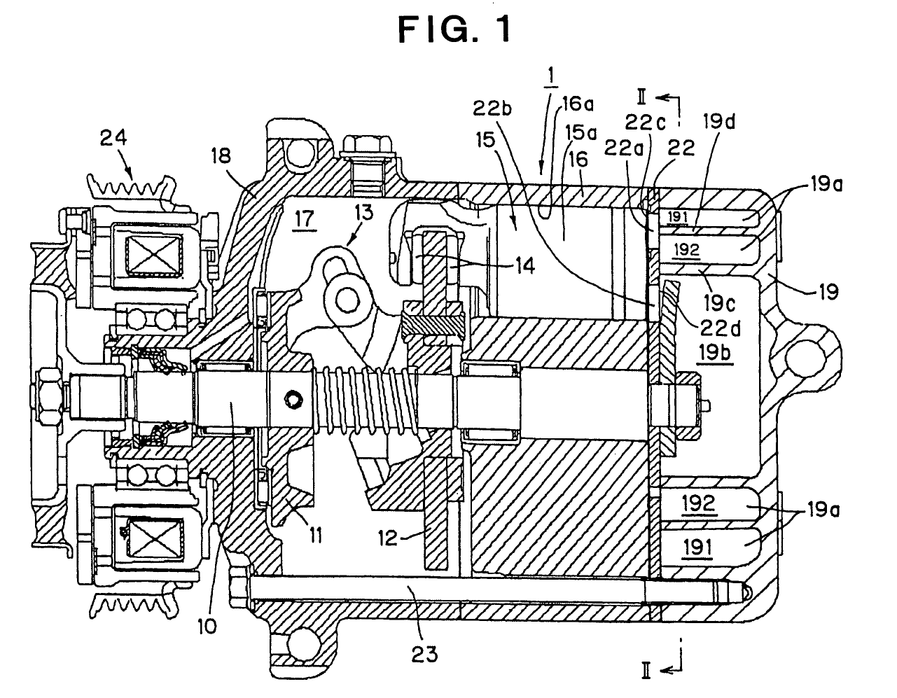

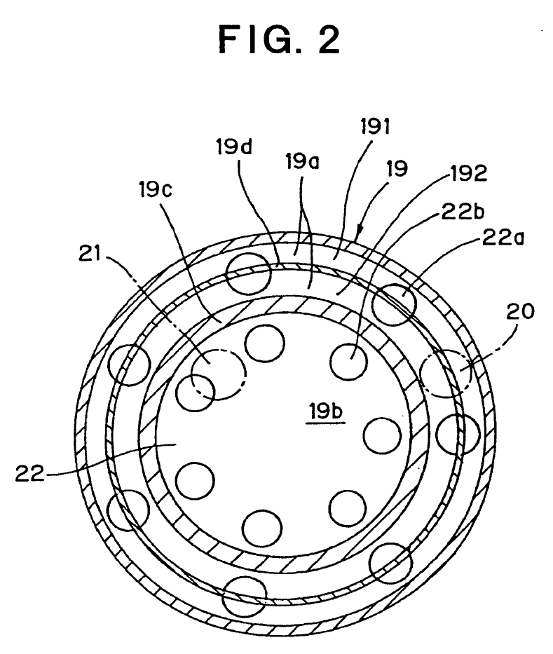

FIGS. 1 and 2 depict an inclined plate-type, variable displacement compressor for use in an air conditioning system (not shown), as a multi-cylinder, reciprocating compressor for an air conditioning system mounted in a vehicle according to an embodiment of the present invention.

In FIG. 1, an inclined plate-type, variable displacement compressor 1, as a multi-cylinder, reciprocating compressor for an air conditioning system mounted in a vehicle comprises a drive shaft 10, a rotor 11 fixed to drive shaft 10, and an inclined plate 12 supported by drive shaft 10, such that inclined plate 12 is adapted to change its inclination angle. Inclined plate 12 is connected to rotor 11 via a link mechanism 13 allowing the change of the inclination angle of inclined plate 12, and inclined plate 12 rotated synchronously with rotor 11 and drive shaft 10.

Each piston 15 engages inclined plate 12 via a pair of shoes 14 sliding on the periphery of inclined plate 12. Head 15a of each piston 15 is inserted into a corresponding cylinder bore 16a formed in a columnar cylinder block 16. A plurality of cylinder bores 16a are disposed at intervals in the circumferential direction around cylinder block 16, and each piston head 15a is inserted into one of cylinder bores 16a, such that piston 15 may reciprocate therein.

A front head 18 having a cup-like shape confronts one end surface of cylinder block 16. Front head 18 forms a crank chamber 17 containing drive shaft 10, rotor 11, and inclined plate 12.

A rear head 19 having a cup-like shape confronts the other end surface of cylinder block 16. Rear head 19 forms an annular suction chamber 19a and a discharge chamber 19b disposed radially inside of suction chamber 19a. Suction chamber 19a and discharge chamber 19b are partitioned from each other by a cylindrical partition wall 19c. Suction chamber 19a is in fluid communication with a suction port 20 formed through the bottom wall of rear head 19, and discharge chamber 19b is in fluid communication with a discharge port 21 formed through the bottom wall of rear head 19. Suction port 20 and discharge port 21 are connected to an air conditioning system mounted in a vehicle (not shown) via a refrigerant circuit.

A valve plate 22 is interposed between cylinder block 16 and rear head 19. Valve plate 22 has suction holes 22a and discharge holes 22b formed therethrough. A suction valve 22c is provided on each suction hole 22a, and a discharge valve 22d is provided on each discharge hole 22b. Suction chamber 19a is in fluid communication with each cylinder bore 16a through suction holes 22a and suction valves 22c. Discharge chamber 19b is in fluid communication with each cylinder bores 16a through discharge holes 22b and discharge valves 22d.

Front head 18, cylinder block 16, valve plate 22, and rear head 19 are fastened together by a plurality of bolts 23 disposed at intervals in the circumferential direction around compressor 1. Drive shaft 10 is supported rotatably by front head 18 and cylinder block 16. One end of drive shaft 10 extends to the exterior of compressor 1 through front head 18. A rotational force is transmitted from an external drive source (not shown) to this exterior end of drive shaft 10 via an electromagnetic clutch 24 attached to front head 18.

As depicted in FIG. 2, suction chamber 19a comprises two chambers: a first chamber 191, located at a radially exterior position, and a second chamber 192, located at a radially interior position (located at a position radially inside of first chamber 191), by an annular suction chamber partition wall 19d continuously extending in the circumferential direction around compressor 1. Both chamber 191 and second chamber 192 are in fluid communication with suction port 20 and with respective suction holes 22a.

In the above-described inclined plate-type, variable displacement compressor 1 for an air conditioning system mounted in a vehicle, the rotation of drive shaft 10 is transmitted to inclined plate 12 via rotor 11 and link mechanism 13. The reciprocating movement of the periphery of inclined plate 12, accompanying the rotation of inclined plate 12, is transmitted to each piston 15 via shoes 14. Head 15a of each piston 15 reciprocates in one of cylinder bores 16a, and refrigerant gas, which flows into cylinder bore 16a through suction port 20, suction chamber 19a, suction hole 22a, and suction valve 22c after circulating in the air conditioning system, is compressed by the reciprocating movement of piston 15. The compressed refrigerant gas is discharged from cylinder bores 16a into discharge chamber 19b through discharge holes 22b and discharge valves 22d. The refrigerant gas discharged into discharge chamber 19b flows into the air conditioning system through discharge port 21.

In such an inclined plate-type, variable displacement compressor 1, annular suction chamber 19a is divided into radially exterior, first chamber 191 and radially interior, second chamber 192, and the circumferential lengths of first and second chambers 191 and 192 are different from each other. Consequently, the frequency of the pulsation of refrigerant generated in suction chamber 19a is dispersed between two frequency values separated from each other as compared with a single frequency value in the known structure of a single, annular suction chamber. By changing the pulsation in suction chamber 19a from a pulsation with a single frequency value to a pulsation with at least two frequency values, the vibration level of the pulsation may be reduced, the levels of the vibration and noise of an air conditioning system ascribed to the pulsation may be reduced, and, ultimately, the noise of the air conditioning system as a whole may be reduced.

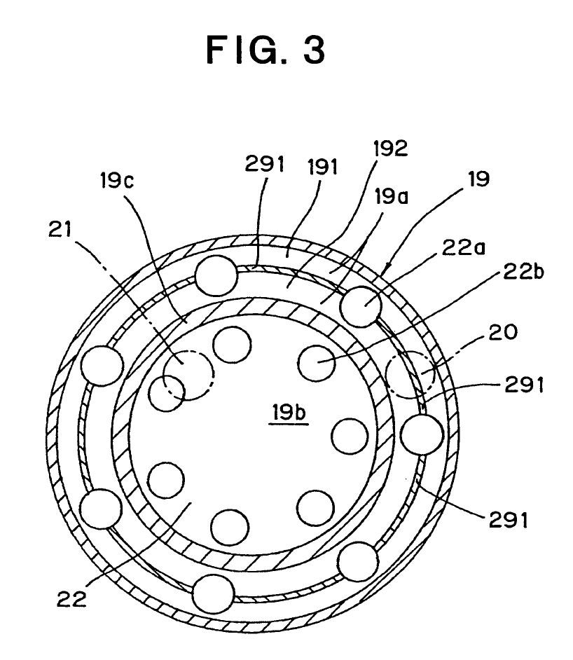

FIG. 3 depicts an inclined plate-type, variable displacement compressor to be connected to an air conditioning system (not shown), as a multi-cylinder, reciprocating compressor for an air conditioning system mounted in a vehicle, according to another embodiment of the present invention. Although suction chamber partition wall 19d continuously extending in the circumferential direction is provided in the above-described embodiment, in this embodiment, suction chamber partition wall 19d is divided into a plurality of partial partition walls 291 extending in the direction of particular wall. In this embodiment, each end of each of the plurality of partial partition walls 291 is disposed at an edge of suction hole 22a.

By dividing suction chamber partition wall 19d into plurality of partial partition walls 291 extending in the direction of partition wall 19d, a plurality of propagation routes for the pulsation directed to suction port 20 are created, a condition similar to the condition, in which a plurality of suction chambers with different circumferential lengths are created. Consequently, the frequency of the pulsation of refrigerant generated in suction chamber 19a is dispersed among many frequency values as compared with a single frequency value in the known structure of a single annular suction chamber. By changing the pulsation in suction chamber 19a from a pulsation with a single frequency value to a pulsation with a plurality of frequency values, the vibration level of the pulsation may be reduced further, the levels of the vibration and noise of an air conditioning system ascribed to the pulsation may be reduced further, and, ultimately, the noise of the air conditioning system as a whole may be reduced further.

In this embodiment, it is preferred that an uneven number of partial partition walls 291 are created. In the depicted embodiment, the number of partial partition walls 291 is set at seven. When suction chamber partition wall 19d is divided into a plurality of partial partition walls 191 extending in the direction of partition wall 19d, a division into an uneven number may achieve a more effective reduction of the vibration level of the pulsation than a division into an even number.

The present invention may be applied not only to an inclined plate-type, compressor, but also to any other multi-cylinder, reciprocating compressor for an air conditioning system mounted in a vehicle, such as a wobble plate-type compressor.

Although embodiments of the present invention have been described in detail herein, the scope of the invention is not limited thereto. It will be appreciated by those skilled in the art that various modifications may be made without departing from the scope of the invention. Accordingly, the embodiments disclosed herein are only exemplary. It is to be understood that the scope of the invention is not to be limited thereby, but is to be determined by the claims which follow.

What is claimed is:

1. A multi-cylinder, reciprocating compressor for an air conditioning system mounted in a vehicle, said compressor comprising:
a columnar cylinder block comprising a plurality of cylinder bores formed therethrough at intervals in a circumferential direction;
a first head formed as a cup-like shape, which confronts one end surface of said cylinder block to form a crank chamber;
a second head formed as a cup-like shape, which confronts the other end surface of said cylinder block;
a drive shaft extending in said crank chamber and through a first wall of said first head;
a valve plate interposed between said cylinder block and said second head;
a discharge chamber formed in said second head and in fluid communication with a discharge port formed on a second wall of said second head and with each of said cylinder bores through a discharge valve and a discharge hole formed through said valve plate; and
a suction chamber disposed in said second head having an annular shape and disposed radially outside of said discharge chamber, which is in fluid communication with a suction port formed through said second wall of said second head and with each of said cylinder bores through a suction valve and a suction hole formed through said valve plate, and which comprises a first chamber and a second chamber, said second chamber is disposed radially inside of said first chamber, and said first and second chambers are separated by a suction chamber partition wall extending in a circumferential direction.

2. The compressor of claim 1, wherein said suction chamber partition wall is comprises a plurality of partial partition walls, each extending in a direction of said suction chamber partition wall.

3. The compressor of claim 2, wherein said plurality of partial partition walls is an uneven number of partial partition walls.

4. The compressor of claim 2, wherein each end of each of said plurality of partial partition walls is disposed at an edge of said suction hole.
